(19) United States
(12) Patent Application Publication
(10) Pub. No.: US 2023/0256065 A1
(43) Pub. Date: Aug. 17, 2023

(54) AUTOLOGOUS STEM CELL VACCINE AND METHODS

(71) Applicants: Elena RUSYN, Marina Del Rey, CA (US); Sean Berman, Beverly Hills, CA (US); Mark Berman, Beverly Hills, CA (US); Elliot Lander, Rancho Mirage, CA (US)

(72) Inventors: Elena RUSYN, Marina Del Rey, CA (US); Sean Berman, Beverly Hills, CA (US); Mark Berman, Beverly Hills, CA (US); Elliot Lander, Rancho Mirage, CA (US)

(21) Appl. No.: 17/673,107

(22) Filed: Feb. 16, 2022

(51) Int. Cl. A61K 39/00; A61P 35/00; A61K 39/12; A61P 37/04; A61K 35/28

(52) U.S. Cl. CPC A61K 39/0011; A61P 35/00; A61K 39/12; A61P 37/04; A61K 35/28; A61K 2035/124

(57) ABSTRACT

The invention provides an immunogenic composition and methods for making and using the composition to generate an immune response. The immunogenic composition comprises stem cells pulsed with an antigen against which an immune response is desired. The stem cells can be autologous mesenchymal stem cells (MSCs), hematopoietic stem cells (HSCs), or stromal vascular fraction (SVF) cells. The cellular vaccine composition finds use in generating an immune response against viral, bacterial and parasitic infections, cancer, and senescent cells.

### FIELD OF THE INVENTION

The invention generally relates to cellular vaccines. More particularly, the invention relates to cellular vaccines comprising autologous stem cells that are pulsed with antigens for generating a desired immunogenic response.

### BACKGROUND

While the vaccination approach is the most effective way of preventing many infectious diseases, traditional vaccine programs are slow to develop and have failed to provide long-term immunity against certain human pathogens.

The unique antigen uptalking and antigen presenting properties of mesenchymal stem cells (MSCs) can provide a new, universal vaccine platform for effective and fast vaccine development for different types of pathogens. MSCs can uptake and present one or several different antigens making them a universal vaccine platform. For example, they can be used with different variants of COVID-19 or as a “senolytic vaccine” for removal of senescent cells and delaying aging and prolonging human lifespan.

To be more successful in using mesenchymal cells as vaccines, MSCs must be invisible to the subject's immune system. Utilizing autologous MSCs as a vaccine platform offers immune tolerability eliminating the risk associated with a recipient-donor mismatch and other possible complications associated with allogeneic stem cells. In addition, it addresses individual variations in post-translational processing of proteins, mimicking natural infection, and can be superior to using allogeneic cells, bacterial, and other expression systems. Autologous MSC have demonstrated success and an excellent safety profile in anti-cancer therapeutic vaccines. Treatments with MSCs and SVF (stromal vascular fraction) cells have excellent safety profiles and are widely used for other purposes.

Thus, autologous mesenchymal stem cells can be a safe, fast and universal platform for designing cellular vaccines.

### SUMMARY OF THE INVENTION

The invention provides an immunogenic composition comprising MSCs, hematopoietic stem cells (HSC), or stromal vascular fraction (SVF) cells that are pulsed with one or more exogenous antigens. In preferred embodiments, the MSCs, HSCs, or SVF cells are obtained from the subject to be treated and are pulsed in vitro with one or more exogenous antigens against which an immune response is desired. The immunogenic composition is administered to the subject one or more times to provide the subject with an effective, adaptive immune response against the antigen thereby treating the infection or disease, or removing the senescent cells with which the antigen is associated.

Without wishing to be bound to any particular theory or mechanism, pulsing MSCs, HSCs, and SVF cells with antigens permits the cells to act as antigen-presenting cells without viral vector or episomal transfection. In addition, the use of autologous cells eliminates the risk of recipient-donor mismatch and other possible complications associated with allogeneic donor cells.

### BRIEF DESCRIPTION OF THE DRAWINGS

FIG. 1 is a process diagram for an embodiment of a method of the invention.

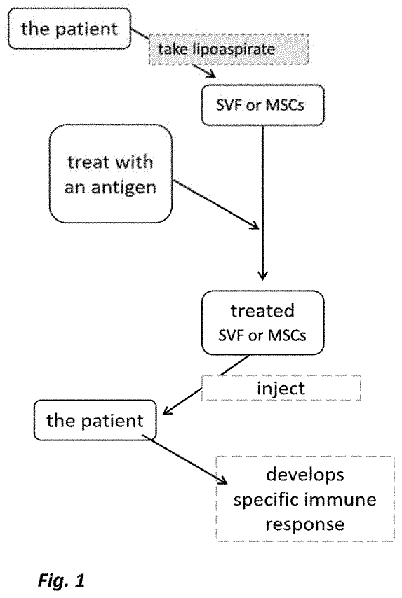

FIG. 2 illustrates competitive ELISA results for serum antibody production with anti-spike recombinant SARS-CoV-2 protein graphed as 450 nm absorbance versus controls and experimental groups using 1:500 serum dilution. Group 1 (G1) are mice that underwent surgical procedure without immunization. Group 3 (G3) are mice that were immunized with SARS-CoV-2 spike protein without pulsed MSCs. Group 5 (G5) are mice that were immunized with autologous MSCs pulsed with SARS-CoV-2 spike protein.

### DETAILED DESCRIPTION

The invention provides an immunogenic composition and methods for making and using the composition for generating an immune response against an antigen of interest for the treatment of infections, diseases, and the removal of senescent cells from the body.

In some embodiments, the immunogenic composition comprises cells that are pulsed with one or more antigens in vitro. One skilled in the art will understand that the terms “pulsed,” “pulsing,” and “pulse” as used herein refer to incubating, culturing, or otherwise contacting cells with one or more antigens under conditions (e.g. in vitro conditions) sufficient for the cells to internalize a quantity of the antigens. Cells that are pulsed with antigen include cells that have internalized a quantity of antigen that is sufficient to generate an immune response when the cells are administered to a subject.

In some embodiments, the cells are pulsed with exogenous antigens. As used herein, the term “exogenous” is used to refer to a substance that is placed into a cell from an external source (e.g., not natural to the cell), including substances such as, for example polypeptides, proteins, recombinant and synthetic peptides and proteins, lipoproteins, glycoproteins, polynucleotides, oligonucleotides, polysaccharides, oligosaccharides, lipids, glycolipids, and other biomolecules. Accordingly, antigens for use with the invention include, but are not limited to, polypeptides, proteins, recombinant and synthetic polypeptides and proteins, lipoproteins, glycoproteins, polynucleotides, oligonucleotides, polysaccharides, oligosaccharides, lipids, glycolipids, and combinations thereof. In a preferred embodiment, cells can be pulsed with one or more exogenous polypeptides, exogenous proteins, or a combination thereof.

In some aspects, the antigens are purified. As used herein, the term “purified” or “pure” refers to a substance (e.g., antigen) that contains less than about 10% impurities. In one embodiment, the term “purified” or “pure” refers to a substance that contains less than about 5% impurities, less than about 2% impurities, or less than about 1% impurities. The term “purified” or “pure” can also refer to a substance that contains about 0% impurities. The term “about,” when used in reference to a quantity, amount, percentage, or other value, means the quantity, amount, percentage, or other value that is referenced, or that varies (plus or minus) by up to 10% of the stated quantity, amount, percentage or other value.

Cells suitable for use with the immunogenic composition of the invention include, but are not limited to, MSCs, HSCs, SVF cells, breast milk stem cells, induced pluripotent stem cells, or combinations thereof. In a preferred embodiment, the cells are autologous with respect to the subject that is to be administered the immunogenic composition. Cells for use with the invention may or may not be expanded for one or more passages before they are pulsed with one or more exogenous antigens.

MSCs for use with the invention may be derived from any tissue that provides cells capable of being pulsed with an antigen and administered to a subject as disclosed herein. Tissues for deriving a suitable source of MSCs include, but are not limited to, adipose, umbilical tissue, umbilical cord blood, placenta, bone marrow, peripheral blood, dermis, periosteum, synovium, skin, hair root, dental pulp, muscle, uterine endometrium, chorionic villus, amniotic fluid, or combinations thereof. In a preferred embodiment, the cells are MSCs that are expanded from a lipoaspirate, from umbilical cord tissue, from umbilical cord blood, or combinations thereof. In another preferred embodiment, the cells are SVF cells obtained from a lipoaspirate. The SVF cells can be obtained from a lipoaspirate and pulsed with one or more exogenous antigens without expanding the cells prior to the pulsing step, wherein the pulsed cells are used as fresh cells at the point of care. Cells for use with the invention can be autologous cells that are collected from the subject to be treated and cryopreserved and banked for later use. In such embodiments, the banked cells can be thawed and pulsed with antigen and administered to the subject. Alternatively, the banked cells can be expanded for one or more passages prior to being pulsed with antigen and administered to the subject. In yet another embodiment, the cells can be expanded and pulsed with exogenous antigens and cryopreserved to provide an array of banked, autologous cells directed to generating an immune response against different pathogens, disorders, diseases, or senescent cells.

In some aspects of the invention, the cells are pulsed with one or more exogenous antigens against which an adaptive, humoral immune response is desired. The exogenous antigens can be viral antigens, bacterial antigens, parasite antigens, cancer cell antigens, senescent cell antigens, or combinations thereof. The antigen can be one or more antigenic polypeptides, one or more antigenic proteins, or a combination thereof. The antigenic polypeptides and/or antigenic proteins can be recombinant or synthetic. Antigenic polypeptides and antigenic proteins for use with the invention can be immunogenic fragments of the antigenic polypeptides or antigenic proteins that are selected for pulsing the cells.

In some embodiments, the invention provides a method of generating an immune response in a subject. The immune response can be generated to treat a targeted infection or targeted disease (e.g., cancer), or to remove senescent cells from the body of subject. The method can be practiced by obtaining MSCs, HSCs and/or SVF cells from the subject, pulsing the cells with one or more exogenous antigens to provide an immunogenic composition that is adapted to generate an immune response against an infection, disease, or senescent cells against which an immune response is desired, and administering the immunogenic composition to the subject. In a preferred embodiment, administering the composition generates a humoral immune response in the subject. The immunogenic composition can be administered to the subject intravenously, subcutaneously, intramuscularly, intranasally, or via suppository. As used herein, the term “subject” refers to mammals, including, without limitation, humans, non-human primates, companion animals (e.g., dogs and cats), rodents (e.g., mice and rats), cows, sheep, pigs, goats, rabbits, and horses.

In some aspects, the method of generating an immune response can include administering an immunogenic composition that comprises cells pulsed with one or more exogenous antigens as disclosed herein, wherein the composition further comprises one or more adjuvants, one or more co-stimulatory molecules, or a combination thereof. Alternatively, the method can comprise administering an immunogenic composition comprising cells pulsed with one or more exogenous antigens as disclosed herein, and administering a separate composition that comprises one or more adjuvants, one or more co-stimulatory molecules, or a combination thereof. The separate composition can be administering simultaneously, or sequentially with the immunogenic composition. Co-stimulatory molecules for use with the invention include, but are not necessarily limited to, cytokines, such as INT-gamma, for example

As used herein, the term “treating” refers to reversing, preventing, alleviating or inhibiting the progress of an infection or disease, or one or more symptoms of an infection, or disease. As used herein, “treating” may also refer to decreasing the probability or incidence of the occurrence of an infection or disease in a subject as compared to an untreated control population, or as compared to the same subject prior to treatment. For example, as used herein, “treating” may refer to preventing an infection or disease, and may include delaying or preventing the onset of an infection or disease, or delaying or preventing the symptoms associated with an infection or disease. As used herein, “treating” may also refer to reducing the severity of an infection or disease, or symptoms associated with such infection or disease prior to affliction with the infection or disease. Such prevention or reduction of the severity of an infection or disease prior to affliction relates to the administration of the composition of the present technology, as described herein, to a subject that is not at the time of administration afflicted with the infection or disease. As used herein “treating” may also further refer to preventing the recurrence of an infection or disease, or of one or more symptoms associated with such infection or disease. The terms “treat,” “therapy,” “treatment,” and “therapeutically,” as used herein, refer to the act of treating as defined herein.

In a preferred embodiment, the invention provides an immunogenic composition for treating Covid 19, wherein the immunogenic composition comprises cells that are pulsed with one or more SARS-CoV-2 (2019-nCoV) antigens, including, but not necessarily limited to, a nucleocapsid protein or fragment thereof, a spike protein or fragment thereof, or combinations thereof. The spike protein or its fragment can be one or more of S1, S1 RBD, and S2. In some embodiments, the immunogenic composition can further include one or more adjuvants, one or more co-stimulatory molecules, or a combination thereof. Alternatively, the immunogenic composition can comprise cells pulsed with one or more SARS-CoV-2 antigens as disclosed herein, which is administered simultaneously with, or in series with, a separate composition comprising one or more adjuvants, one or more co-stimulatory molecules, or a combination thereof. Co-stimulatory molecules for use with the invention include, but are not necessarily limited to, cytokines, such as INT-gamma, for example.

In another embodiment, the invention provides an immunogenic composition for treating viral infections, wherein the immunogenic composition comprises cells that are pulsed with one or more viral antigens. Suitable viral antigens for use with the invention include, without limitation, antigens from other coronaviruses in addition to 2019-nCoV, adenovirus antigens, retroviral antigens, rotavirus antigens, flavivirus antigens, hepacivirus antigens, papillomavirus antigens, hepadnavirus antigens, parvovirus antigens, pox virus antigens, Epstein-Barr virus antigens, cytomegalovirus antigens, herpes simplex virus antigens, varicella zoster virus antigens, roseolovirus antigens, Filovirus antigens, paramyxovirus antigens, orthomyxovirus antigens, rhabdovirus antigens, arenavirus antigens, human enterovirus antigens, hepatitis A virus antigens, human rhinovirus antigens, rubella virus antigens, polio virus antigens, and combinations thereof.

In another embodiment, the invention provides an immunogenic composition for treating a bacterial infection, wherein the immunogenic composition comprises cells that are pulsed with one or more bacterial antigens. Bacterial antigens for use with the invention include, but are not necessarily limited to, Bacillus antigens, Bordetella antigens, Borrelia antigen, Brucella antigens, Burkholderia antigens, Campylobacter antigens, Chlamydia antigens, Chlamydophila antigens, Clostridium antigens, Corynebacterium antigens, Enterococcus antigens, Escherichia antigens, Francisella antigens, Haemophilus antigens, Helicobacter antigens, Legionella antigens, Leptospira antigens, Listeria antigens, Mycobacterium antigens, Mycoplasma antigens, Neisseria antigens, Pseudomonas antigens, Rickettsia antigens, Salmonella antigens, Shigella antigens, Staphylococcus antigens, Streptococcus antigens, Treponema antigens, Vibrio antigens, Yersinia antigens, and combinations thereof.

In another embodiment, the invention provides an immunogenic composition for treating a parasitic infection, wherein the immunogenic composition comprises cells that are pulsed with one or more parasite antigens. The parasite antigens can be antigens from a single celled parasite, a multicellular parasite, or a combination thereof. Suitable parasite antigens for use with the invention include, but are not necessarily limited to, Acanthamoeba antigens, Anisakis antigens, Ascaris antigens, lumbricoides antigens, Balantidium coli antigens, Cestoda (tapeworm) antigens, chiggers antigens, Cochliomyia hominivorax antigens, Entamoeba histolytica antigens, Fasciola hepatica antigens, Giardia lamblia antigens, hookworm antigens, Leishmania antigens, Linguatula serrata antigens, liver fluke antigens, Loa boa antigens, Paragonimus (lung fluke) antigens, pinworm antigens, Plasmodium falciparum antigens, Schistosoma antigens, Strongyloides stercoralis antigens, tapeworm antigens, Toxoplasma gondii antigens, Trypanosoma antigens, whipworm antigens, Wuchereria bancrofti antigens, and combinations thereof.

In another embodiment, the invention provides an immunogenic composition for treating cancer, wherein the immunogenic composition comprises cells that are pulsed with one or more cancer antigens. Cancer antigens suitable for use with the invention include, but are not necessarily limited to, antigens derived from gut carcinoma, colorectal cancer, metastatic colorectal cancer, colorectal carcinoma, lung carcinoma (e.g. lung squamous carcinoma and non-small cell lung carcinoma), colorectal, gastric cancer, endometrial carcinoma, melanoma, chronic myeloid leukemia, head and neck squamous cell carcinoma, acute lymphoblastic leukemia, acute myelogenous leukemia, chronic lymphocytic leukemia, renal cell carcinoma, ovarian cancer, endometrial cancer, pancreatic adenocarcinoma, bladder tumor, pituitary tumor, breast cancer, prostate cancer, prostate carcinoma, liver cancer, thyroid cancer, CNS cancer, stomach cancer, skin cancer, hematopoietic malignancies, lymph node cancer, adrenal glands cancer, bone marrow cancer, retinal cancer, sarcoma, thymus cancer, spleen cancer, and combinations thereof.

Suitable cancer antigens for use with the invention further include, but are not necessarily limited to, BAGE-1, CT37/FMR1NB, Cyclin-A1, D393-CD20n, GAGE-1,2,8, GAGE-3,4,5,6,7, GnTV, HERV-E, HERV-K-MEL, KK-LC-1, KM-HN-1, LAGE-1, LRPAP1, LY6K, MAGE-A1, MAGE-A10, MAGE-A2, MAGE-A3, MAGE-A4, MAGE-A6, MAGE-A9, MAGE-C1, MAGE-C2, Mucin, NA88-A, NY-ESO-1/LAGE-2, SAGE, Sp17, SSX-2, SSX-4, TAG-1, TAG-2, TRAG-3, TRP2-INT2, XAGE-1b/GAGED2a, adipophilin, AIM-2, ALDH1A1, alpha-foetoprotein (AFP), BCLX (L), BING-4, CALCA, CD274, CD45, CPSF, cyclin D1, DKK1, ENAH (hMena), EpCAM, EphA3, EZH2, FGFS, G250/MN/CAIX, glypican-3, HEPACAM, Hepsin, HER-2/neu, HLA-DOB, HLA-G, HSPH1, IDOL IGF2B3, IL13Ralpha2, IMP-3, Intestinal carboxyl esterase, Kallikrein 4, KIF20A, Lengsin, M-CSF, MCSP, mdm-2, Meloe, Midkine, MMP-2, MMP-7, MUC1, MUCSAC, nectin-4, p53, PAXS, PBF, PLAC1, PRAME, PSMA, RAGE-1, RGSS, RhoC, RNF43, RU2AS, secernin 1, SOX10, STEAP1, survivin, telomerase, TPBG, VEGF, WT1, CEA, gp100/Pme117, Melan-A/MART-1, NY-BR-1, OA1, PAP, PSA, RAB38/NY-MEL-1, TRP-1/gp75, TRP-2, tyrosinase, alpha-actinin-4, AP2S1, ARTC1, B-RAF, BCR-ABL fusion protein (b3a2), beta-catenin, CASP-5, CASP-8, Cdc27, CDK12, CDK4, CDKN2A, CLPP, COA-1, CSNK1A1, dek-can fusion protein, EFTUD2, Elongation factor 2, ETV6-AML1 fusion protein, FLT3-ITD, FN1, FNDC3B, GAS7, GPNMB (glycoprotein nonmetastatic melanoma protein B), HAUS3, HLA-A11, HLA-A2, HSDL1, hsp70-2, K-ras, KIAAO205, LDLR-fucosyltransferaseAS fusion protein, MART2, MATN, ME1, MUM-1, MUM-2, MUM-3, Myosin class I, N-ras, neo-PAP, NFYC, OGT, OS-9, pml-RARalpha fusion protein, PPP1R3B, PRDXS, PTPRK, RBAF600, SIRT2, SNRPD1, SYT-SSX1 or -SSX2 fusion protein, TGF-betaRII, TP53, Triosephosphate isomerase, and combinations thereof.

Other suitable antigens for use with the invention can include, but are not necessarily limited to, influenza hemagglutinin 1 (HA1), hemagglutinin 2 (HA2), influenza neuraminidase (NA), Lassa virus (LASV) glycoprotein 1 (gp1), LASV glycoprotein 2 (gp2), LASV nucleocapsid-associated protein (NP), LASV L protein, LASV Z protein, SARS virus S protein, Ebola virus GP2, measles virus fusion 1 (F1) protein, HIV-1 transmembrane (TM) protein, HIV-1 glycoprotein 41 (gp41), HIV-1 glycoprotein 120 (gp120), hepatitis C virus (HCV) envelope glycoprotein 1 (E1), HCV envelope glycoprotein 2 (E2), HCV nucleocapsid protein (p22), West Nile virus (WNV) envelope glycoprotein (E), Japanese encephalitis virus (JEV) envelope glycoprotein (E), yellow fever virus (YFV) envelope glycoprotein (E), tick-borne encephalitis virus (TBEV) envelope glycoprotein (E), hepatitis G virus (HGV) envelope glycoprotein 1 (E1), respiratory syncytial virus (RSV) fusion (F) protein, herpes simplex virus 1 (HSV-1) gD protein, HSV-1 gG protein, HSV-2 gD protein, HSV-2 gG protein, hepatitis B virus (HBV) core protein, Epstein-Barr virus (EBV) glycoprotein 125 (gp125), bacterial outer membrane protein assembly factor BamA, bacterial translocation assembly module protein TamA, bacterial polypeptide-transport associated protein domain protein, bacterial surface antigen D15, anthrax protective protein, anthrax lethal factor, anthrax edema factor, Salmonella typhii S1Da, Salmonella typhii S1Db, cholera toxin, cholera heat shock protein, Clostridium botulinum antigen S, botulinum toxin, Yersinia pestis F1, Yersina pestis V antigen, Yersinia pestis YopH, Yersinia pestis YopM, Yersinia pestis YopD, Yersinia pestis plasminogen activation factor (Pla), Plasmodium circumsporozoite protein (CSP), Plasmodium sporozoite surface protein (SSP2/TRAP), Plasmodium liver stage antigen 1 (LSAT), Plasmodium exported protein 1 (EXP 1), Plasmodium erythrocyte binding antigen 175 (EBA-175), Plasmodium cysteine-rich protective antigen (cyRPA), Plasmodium heat shock protein 70 (hsp70), Schistosoma Sm29, and Schistosoma signal transduction protein 14-3-3, GPNMB (glycoprotein nonmetastatic melanoma protein B), and combinations thereof.

In another embodiment, the invention provides an immunogenic composition for senolytic therapy, wherein the immunogenic composition comprises cells that are pulsed with one or more antigens that are expressed by senescent cells. In such embodiments, the immunogenic composition elicits a humoral immune response against senescent cells which express the targeted senescent cell antigen thereby inducing a humoral immune response that removes senescent cells from the body of the subject. Senescent cell antigens suitable for use with the invention include, but are not necessarily limited to, GPNMB (glycoprotein nonmetastatic melanoma protein B), and other candidates from genes and pathways regulated in aging and age-related diseases. The senescent cell antigens can be biomarker candidates from different “hallmark of aging” pathways, such as inflammation, mitochondria and apoptosis, calcium homeostasis, fibrosis, NMJ (neuromuscular junctions) and neurons, cytoskeleton proteins, and hormones. Without wishing to be bound to any particular theory or mechanism, the accumulation of senescent cells in the body can been associated with aging and aging-related diseases, such as Alzheimer's disease and atherosclerosis. Thus, the immunogenic composition disclosed herein can be administered to treat these and other age-related diseases, and as a means for promoting the health and longevity of a subject, particularly subjects of advanced age.

In some embodiments, the invention provides a method of making an immunogenic composition for generating a desired immune response. The method can be practiced by providing at least one of MSCs, HSCs, and SVF cells, and pulsing the cells with one or more exogenous antigens as disclosed herein. The method of making the immunogenic composition can further include combining the cells with a pharmaceutically acceptable carrier for administering the cells according to one or more administration routes as disclosed herein. In some aspects of the invention, the pharmaceutically acceptable carrier is an artificial pharmaceutically acceptable carrier. In further aspects, the pharmaceutically acceptable carrier is a nanocarrier. Suitable pharmaceutically acceptable carriers for use with the invention include, but are not necessarily limited to, those disclosed in the following references, the entire contents of which are incorporated herein by reference for all purposes: Remington: The Science and Practice of Pharmacy, 19^{th} Ed (Easton, Pa.: Mack Publishing Company, 1995); Hoover, John E., Remington's Pharmaceutical Sciences, (Easton, Pa.: Mack Publishing Co 1975); Liberman, H. A. and Lachman, L., Eds., Pharmaceutical Dosage Forms (New York, N.Y.: Marcel Decker 1980); and Pharmaceutical Dosage Forms and Drug Delivery Systems, Seventh Ed (Lippincott Williams & Wilkins 1999). In some embodiments, the method of making the immunogenic composition can include combining the pulsed cells with one or more adjuvants, one or more co-stimulatory molecules, or a combination thereof.

### EXAMPLES

### Example 1

### Protocol for SVF (Stromal Vascular Fraction) Cellular Vaccine

Do local anesthesia in the area for liposuction

Do sterile prep

Do liposuction procedure, aspirate adipose tissue

Apply Bacitracin ointment, compression bandage and a band-aid

Purify SVF cells

Count cells, determine viability and uniformity

Test for endotoxin and sterility

Cryopreserve or use immediately

Prepare antigen mix

Add antigen mix to the cells

Incubate

Deliver the primed autologous cells to the patient's body:
a. Intravenous
b. Intramuscular
c. Subcutaneous
d. Intranasal
e. Rectal suppositories

### Antigens

In a preferred embodiment, one or more fragments of COVID 19, SARS-CoV-2 (2019-nCoV) spike recombinant protein are used as the antigen. However, the method can be used with other viral antigenic proteins, as well as bacterial antigenic proteins, parasite antigenic proteins, cancer antigenic proteins, and senescent cell antigens.

### Protocol for Adipose-Derived MSC Cellular Vaccine

MSCs are a fraction of SVF cells selected for their ability to adhere to plastic and grow in expansion media. Interventions using MSCs are performed as with SVF cells above, except that SVF cells are grown in culture so that adherent MSCs can be isolated and expanded in culture. Isolated and expanded MSCs can be pulsed with antigen and administered, or cryopreserved and later pulsed with antigen and administered.

### Protocol for Umbilical Cord-Derived MSC Cellular Vaccine

The protocol for preparing umbilical cord-derived MSCs is the same as the adipose-derived MSCs, except that the cells are sourced from umbilical cord tissue. Umbilical cord cells are grown in culture and adherent MSC are isolated and expanded. The expanded cells can be pulsed with antigen and administered, or cryopreserved and later pulsed with antigen and administered.

### Example 2

### Experimental Results

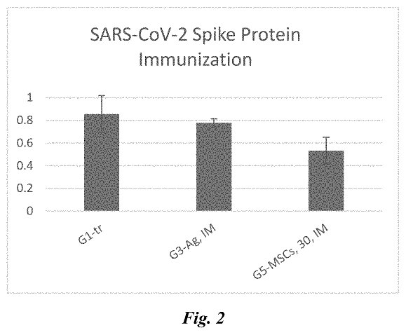

A group of 6 male mice that underwent a single intramuscular immunization procedure with 1 million autologous MSCs pulsed with 7 μg SARS-CoV-2 spike recombinant protein antigen demonstrated a significant neutralizing antibody response compared to a control group that received only SARS-CoV-2 spike protein antigen without MSCs, and a control group of naïve mice that received a surgical autologous fat extraction procedure only (FIG. 2).

Excision of Visceral Adipose Tissue from Live Mice

### Animals

Young adult (6-8 weeks old) male Swiss Webster mice were individually housed under controlled conditions (12:12 light-dark cycle, 24-25% humidity, and 22° C.). A group of 6 mice underwent an excision of visceral peri-gonadal adipose tissue and were kept alive. A primary autologous MSC cell culture was established for each mouse. One million MSCs were pulsed with 7 micrograms of spike protein and 100 microliters was administered with a 1 ml syringe via intramuscular injection. Sera samples were collected on day 17 post-immunization. The group of 6 mice that underwent surgical procedure only and the group of 6 mice that underwent surgical procedure and were intramuscularly injected with antigen without MSCs served as control groups.

Procedures were approved by the Institutional Animal Care and Use Committee (IACUC) of the California State University, Northridge and in accordance with the Guide for the Care and Use of Laboratory Animals from the National Research Council (Eighth Edition, 2011).

Excision of Visceral Adipose Tissue from Live Mice

Following anesthetization with isoflurane, surgeries were performed through a midventral abdominal incision.

The testes were visualized, and the attached peri-gonadal fat pads were separated from surrounding tissue and bilaterally excised. Because the testicular artery originates from the epididymal arteries, care was taken not to disrupt the major vasculature within fat tissues (i.e., in order to preserve testes vasculature). No more than ˜60% of fat was removed in the surgery, totaling ˜1 g. The abdominal incision was closed by suturing the peritoneum, muscle, and skin with absorbable sutures.

### Primary MSCs Culture

Excised adipose tissue were shipped overnight to an FDA-registered reliable stem cell bank where tissues were processed under a well-established protocol that fulfills the criteria of Good Manufacturing Practice (GMP) for the production of clinical-grade MSCs. The MSCs produced under this protocol were evaluated considering the criteria recommended by the international society for cell therapy (ISCT).

Briefly, specimens were cut into smaller bits, subjected to enzymatic digestion with collagenase, and placed in sterile flasks containing complete MSC culture medium. After 5-7 days primary cell cultures were established for each mouse and cells were cryopreserved in early passage in the amount of one million cells per cryovial.

### Immunization Procedure

One million autologous MSCs were pulsed with 7 μg SARS-CoV-2 (2019-nCoV) spike RBD recombinant protein (Sino Biological, LLC Cat No. 40592-VNAH) for 30 minutes in 100 microliters of a complete cell culture media at 37° C. and subsequently injected intramuscularly into the same animal from which they were generated.

### Serum Sample Collection

17 days post-immunization, blood was collected from the tail vein (˜200 μl). After 20 minutes of incubation at room temperature, samples were spined at 3000 rpm and serum was transferred to a new vial.

### Competitive ELISA Assay

The principle of the assay is competitive ELISA. The SARS-CoV-2 (2019-nCoV) neutralizing antibodies in the samples compete with ACE2-His (Cat: 10108-H08B) for the binding site on immobilized SARS-CoV-2 S Protein RBD. The signal color becomes lighter as the content of SARS-CoV-2 inhibitor increases.

The microplate was pre-coated with SARS-CoV-2 (2019-nCoV) Spike RBD-mFc recombinant protein (Sino Biological LLC, China) at the concentration 2 μg/ml. SARS-CoV-2 inhibitor (neutralizing antibodies) served as a control. Serum samples were diluted 1:500 and added to wells at the same time with human ACE2 and incubated for 1 hour at room temperature. The binding of human ACE2 (His Tag) to spike protein was measured by using anti-His tag Ab (HRP) followed by adding a substrate and stop solution. The optical density of each well was determined within 15 minutes, using a microplate reader set to 450 nm.

1. An immunogenic composition, comprising cells having at least one exogenous antigen, wherein said cells comprise one or more cells selected from the group consisting of mesenchymal stem cells (MSCs), hematopoietic stem cells (HSCs), stromal vascular fraction (SVF) cells, breast milk stem cells (BSCs), and induced pluripotent stem cells (iPSCs).

2. The immunogenic composition of claim 1, wherein said MSCs comprise MSCs selected from the group consisting of SVF MSCs, perinatal tissue MSCs, and a combination thereof.

3. The immunogenic composition of claim 2, wherein said perinatal tissue MSCs are umbilical cord blood MSCs, umbilical cord tissue MSCs, or a combination thereof.

4. The immunogenic composition of claim 1, wherein said cells comprise a plurality of exogenous antigens.

5. The immunogenic composition of claim 1, wherein said cells comprise between about 2 and about 100 exogenous antigens.

6. The immunogenic composition of claim 1, wherein said cells are pulsed with said at least one exogenous antigen.

7. The immunogenic composition of claim 1, wherein said at least one exogenous antigen comprises a polypeptide, a protein, or a combination thereof.

8. The immunogenic composition of claim 7, wherein at least one of said polypeptide and said protein is recombinant.

9. The immunogenic composition of claim 1, wherein said at least one exogenous antigen is purified.

10. The immunogenic composition of claim 1, wherein said at least one exogenous antigen comprises one or more antigens selected from the group consisting of a pathogen antigen, a cancer antigen, a senescent cell antigen, and combinations thereof.

11. The immunogenic composition of claim 10, wherein said pathogen antigen comprises antigens from a plurality of pathogens.

12. The immunogenic composition of claim 10, wherein said pathogen antigen is selected from the group consisting of a viral antigen, a bacterial antigen, a parasite antigen, and combinations thereof.

13. The immunogenic composition of claim 12, wherein said viral antigen comprises an antigen selected from the group consisting of: a coronavirus antigen; an adenovirus antigen; a retroviral antigen; a rotavirus antigen; a flavivirus antigen; a hepacivirus antigen; a papillomavirus antigen; a hepadnavirus antigen; parvovirus antigen; a pox virus antigen; an Epstein-Barr virus antigen; a cytomegalovirus antigen; a herpes simplex virus antigen; a varicella zoster virus antigen; a roseolovirus antigen; a filovirus antigen; a paramyxovirus antigen; an orthomyxovirus antigen; a rhabdovirus antigen; an arenavirus antigen; an human enterovirus antigen; a hepatitis A virus antigen; a human rhinovirus antigen; a rubella virus antigen; a polio virus antigen; and combinations thereof.

14. The immunogenic composition of claim 13, wherein said corona virus antigen comprises at least one SARS-CoV-2 (2019-nCoV) antigen.

15. The immunogenic composition of claim 14, wherein said at least one SARS-CoV-2 (2019-nCoV) antigen includes a nucleocapsid protein, a spike protein, or a combination thereof.

16. The immunogenic composition of claim 15, wherein said spike protein comprises a spike protein selected from the group consisting of: S1; S1 RBD; S2; and combinations thereof.

17. The immunogenic composition of claim 12, wherein said bacterial antigen comprises a bacterial antigen selected from the group consisting of: a Bacillus antigen, a Bordetella antigen; a Borrelia antigen; a Brucella antigen; a Burkholderia antigen; a Campylobacter antigen; a Chlamydia antigen; a Chlamydophila antigen; a Clostridium antigen; a Corynebacterium antigen; an Enterococcus antigen; an Escherichia antigen; a Francisella antigen; a Haemophilus antigen; a Helicobacter antigen; a Legionella antigen; a Leptospira antigen; a Listeria antigen; a Mycobacterium antigen; a Mycoplasma antigen; a Neisseria antigen; a Pseudomonas antigen; a Rickettsia antigen; a Salmonella antigen; a Shigella antigen; a Staphylococcus antigen; a Streptococcus antigen; a Treponema antigen; a Vibrio antigen; a Yersinia antigen; and combinations thereof.

18. The immunogenic composition of claim 12, wherein said parasite antigen is a single celled parasite antigen, a multicellular parasite antigen, or a combination thereof.

19. The immunogenic composition of claim 12, wherein said parasite antigen comprises a parasite antigen selected from the group consisting of: an Acanthamoeba antigen; an Anisakis antigen; an Ascaris lumbricoides antigen; a Balantidium coli antigen; a Cestoda (tapeworm) antigen; a Chiggers antigen; a Cochliomyia hominivorax antigen; a Entamoeba histolytica antigen; a Fasciola hepatica antigen; a Giardia lamblia antigen; a hookworm antigen; a Leishmania antigen; a Linguatula serrata antigen; a liver fluke antigen; a Loa boa antigen; a Paragonimus (lung fluke) antigen; a pinworm antigen; a Plasmodium falciparum antigen; a Schistosoma antigen; a Strongyloides stercoralis antigen; a Tapeworm antigen; a Toxoplasma gondii antigen; a Trypanosoma antigen; a whipworm antigen; a Wuchereria bancrofti antigen; and combinations thereof.

20. The immunogenic composition of claim 10, wherein said cancer antigen comprises an antigen from a cancer selected from the group consisting of: gut carcinoma; colorectal cancer; metastatic colorectal cancer; colorectal carcinoma; lung carcinoma; colorectal; gastric; and endometrial carcinoma; melanoma; chronic myeloid leukemia; head and neck squamous cell carcinoma; acute lymphoblastic leukemia; acute myelogenous leukemia; chronic lymphocytic leukemia; renal cell carcinoma; ovarian cancer; endometrial cancer; pancreatic adenocarcinoma; bladder tumor; pituitary tumor; breast cancer; prostate cancer; prostate carcinoma; liver cancer; thyroid cancer; CNS cancer; stomach cancer; skin cancer; hematopoietic malignancies; lymph node cancer; adrenal glands cancer; bone marrow cancer; retinal cancer; sarcoma; thymus cancer; spleen cancer; and combinations thereof.

21. The immunogenic composition of claim 20, wherein said lung cancer is lung squamous carcinoma or non-small cell lung carcinoma.

22. The immunogenic composition of claim 10, wherein said cancer antigen comprises an antigen selected from the group consisting of: BAGE-1; CT37/FMR1NB; Cyclin-A1; D393-CD20n; GAGE-1;2;8; GAGE-3,4,5,6,7; GnTV; HERV-E; HERV-K-MEL; KK-LC-1; KM-HN-1; LAGE-1; LRPAP1; LY6K; MAGE-A1; MAGE-A10; MAGE-A2; MAGE-A3; MAGE-A4; MAGE-A6; MAGE-A9; MAGE-C1; MAGE-C2; Mucin; NA88-A; NY-ESO-1/LAGE-2; SAGE; Sp17; SSX-2; SSX-4; TAG-1; TAG-2; TRAG-3; TRP2-INT2; XAGE-1b/GAGED2a; adipophilin; AIM-2; ALDH1A1; alpha-foetoprotein (AFP); BCLX (L); BING-4; CALCA; CD274; CD45; CPSF; cyclin D1; DKK1; ENAH (hMena); EpCAM; EphA3; EZH2; FGF5; G250/MN/CAIX; glypican-3; HEPACAM; Hepsin; HER-2/neu; HLA-DOB; HLA-G; HSPH1; IDO1; IGF2B3; IL13Ralpha2; IMP-3; Intestinal carboxyl esterase; Kallikrein 4; KIF20A; Lengsin; M-CSF; MCSP; mdm-2; Meloe; Midkine; MMP-2; MMP-7; MUC1; MUCSAC; nectin-4; p53; PAXS; PBF; PLAC1; PRAME; PSMA; RAGE-1; RGSS; RhoC; RNF43; RU2AS; secernin 1; SOX10; STEAP1; survivin; telomerase; TPBG; VEGF; WT1; CEA; gp100/Pme117; Melan-A/MART-1; NY-BR-1; OA1; PAP; PSA; RAB38/NY-MEL-1; TRP-1/gp75; TRP-2; tyrosinase; alpha-actinin-4; AP2S1; ARTC1; B-RAF; BCR-ABL fusion protein (b3a2); beta-catenin; CASP-5; CASP-8; Cdc27; CDK12; CDK4; CDKN2A; CLPP; COA-1; CSNK1A1; dek-can fusion protein; EFTUD2; Elongation factor 2; ETV6-AML1 fusion protein; FLT3-ITD; FN1; FNDC3B; GAS7; GPNMB; HAUS3; HLA-A11; HLA-A2; HSDL1; hsp70-2; K-ras; KIAAO205; LDLR-fucosyltransferaseAS fusion protein; MART2; MATN; ME1; MUM-1; MUM-2; MUM-3; Myosin class I; N-ras; neo-PAP; NFYC; OGT; OS-9; pml-RARalpha fusion protein; PPP1R3B; PRDXS; PTPRK; RBAF600; SIRT2; SNRPD1; SYT-SSX1 or -SSX2 fusion protein; TGF-betaRll; TP53; Triosephosphate isomerase; and combinations thereof.

23. The immunogenic composition of claim 1, wherein said at least one exogenous antigen comprises a protein selected from the group consisting of: influenza hemagglutinin 1 (HA1); hemagglutinin 2 (HA2); influenza neuraminidase (NA); Lassa virus (LASV) glycoprotein 1 (gp1); LASV glycoprotein 2 (gp2); LASV nucleocapsid-associated protein (NP); LASV L protein; LASV Z protein; SARS virus S protein; Ebola virus GP2; measles virus fusion 1 (F1) protein; HIV-1 transmembrane (TM) protein; HIV-1 glycoprotein 41 (gp41); HIV-1 glycoprotein 120 (gp120); hepatitis C virus (HCV) envelope glycoprotein 1 (E1); HCV envelope glycoprotein 2 (E2); HCV nucleocapsid protein (p22); West Nile virus (WNV) envelope glycoprotein (E); Japanese encephalitis virus (JEV) envelope glycoprotein (E); yellow fever virus (YFV) envelope glycoprotein (E); tick-borne encephalitis virus (TBEV) envelope glycoprotein (E); hepatitis G virus (HGV) envelope glycoprotein 1 (E1); respiratory syncytial virus (RSV) fusion (F) protein; herpes simplex virus1 (HSV-1) gD protein; HSV-1 gG protein; HSV-2 gD protein; HSV-2 gG protein; hepatitis B virus (HBV) core protein; Epstein-Barr virus (EBV) glycoprotein 125 (gp125); bacterial outer membrane protein assembly factor BamA; bacterial translocation assembly module protein TamA; bacterial polypeptide-transport associated protein domain protein; bacterial surface antigen D15; anthrax protective protein; anthrax lethal factor; anthrax edema factor; Salmonella typhii S1Da; Salmonella typhii S1Db; cholera toxin; cholera heat shock protein; Clostridium botulinum antigen S; botulinum toxin; Yersinia pestis F1; Yersinia pestis V antigen; Yersinia pestis YopH; Yersinia pestis YopM; Yersinia pestis YopD; Yersinia pestis plasminogen activation factor (Pla); Plasmodium circumsporozoite protein (CSP); Plasmodium sporozoite surface protein (SSP2/TRAP); Plasmodium liver stage antigen 1 (LSAT); Plasmodium exported protein 1 (EXP 1); Plasmodium erythrocyte binding antigen 175 (EBA-175); Plasmodium cysteine-rich protective antigen (cyRPA); Plasmodium heat shock protein 70 (hsp70); Schistosoma Sm29; and Schistosoma signal transduction protein 14-3-3; and combinations thereof.

24. The immunogenic composition of claim 10, wherein said senescent cell antigen comprises GPNMB (glycoprotein nonmetastatic melanoma protein B).

25. The immunogenic composition of claim 1, wherein at least one of said MSCs, said HSCs, and said SVF cells are substantially free of exogenous polynucleotides.

26. The immunogenic composition of claim 1, wherein said cells are substantially free of episomes.

27. The immunogenic composition of claim 1, wherein said immunogenic composition further comprises a pharmaceutically acceptable carrier.

28. The immunogenic composition of claim 27, wherein said pharmaceutically acceptable carrier is an artificial pharmaceutically acceptable carrier.

29. The immunogenic composition of claim 27, wherein said pharmaceutically acceptable carrier is adapted for at least one of intravenous administration, subcutaneous administration, intramuscular administration, intranasal administration, and suppository administration.

30. A method of generating an immune response, comprising administering to a subject an effective amount of the immunogenic composition of claim 1.

31. The method of claim 30, wherein said cells are autologous with respect to said subject.

32. The method of claim 30, wherein said immunogenic composition is administered intravenously, subcutaneously, intramuscularly, intranasally, by suppository, or combinations thereof.

33. The method of claim 30, wherein said immunogenic composition is administered to said subject one or more times.
